(19) United States
(12) Patent Application Publication
(10) Pub. No.: US 2026/0124348 A1
(43) Pub. Date: May 7, 2026

(54) BREAST PUMP

(71) Applicant: AMPLIM, LLC, Portola Valley, CA (US)

(72) Inventor: Peng Lim, Portola Valley, CA (US)

(21) Appl. No.: 18/937,499

(22) Filed: Nov. 5, 2024

(51) Int. Cl. A61M 1/06

(52) U.S. Cl. CPC A61M 1/062; A61M 2202/0014; A61M 2202/04; A61M 2205/0216; A61M 2210/1007

(57) ABSTRACT

An easy to assemble breast pump system with an effective, pain-free, and easy to operate manual pumping mechanism that allows for either one-handed or two-handed operation is disclosed for use in stimulating lactation, particularly of the female human breast. The breast milk pump system is wearable including a pump/collector fitted within a frame and the pump/collector is deformable to generate negative air pressure to facilitate breast milk collection. The breast pump system includes a hollow internal chamber having a breast receiving area adapted to engage at least the mammary papilla and areola area and means for pressurizing and depressurizing said chamber when in engagement with the breast. The breast receiving area includes an aperture large enough for only the mammary papilla and areola area to extend through.

### TECHNICAL FIELD

The present disclosure relates generally to devices, systems, and techniques for a breast pump system; one implementation of the system is a wearable breast pump system for extracting milk from a breast and, more particularly, pertains to a new and improved method and apparatus for extracting milk from the breast.

### BACKGROUND

Breast pumps are convenient for nursing mothers, because, among other things, they allow the nursing mother to draw off and store breast milk to feed to their child at a later time. Additionally, for some mothers, breast pumps are a necessity, particularly when the child has nursing difficulties, or if the mother has problems with excessive or deficient milk production, or cannot empty completely. Some mothers also require breast pumps in the event of soreness or injury of the mammary papilla and/or areola.

Manually operated breast pumps are commonplace; however, they typically require the use of both hands to pump a single breast, for example one hand is used to hold the breast pump in place, and the other to drive the pump. There are also manually operated breast pumps that can be operated with one hand, as by using a lever-type drive mechanism. These require some manual effort to operate. Manually operated breast pumps also can put pressure on the mammary papilla and/or areola area, causing pain and soreness from use.

### SUMMARY

According to one aspect of the disclosure a wearable breast pump is provided. The breast pump includes a deformable container with a front portion and a rear portion and a frame. The rear portion of the deformable container shaped to contour a user's breast. The frame being configured to engage with the front portion of the deformable container.

In addition to one or more of the features described herein, or as an alternative, further embodiments of the deformable container may include, a stopper on a top end of the deformable container, a flange adjacent to the stopper, a breast receiving surface on the rear portion of the deformable container that comes into contact with the user's breast, an aperture formed in the breast receiving surface, and an hollow internal chamber. A user's mammary papilla and/or areola area is inserted at least partially into the aperture and the expressed milk is collected in the hollow internal chamber. In addition to one or more of the features described herein, or as an alternative, further embodiments of the deformable container may include, the deformable container being squeezable to generate negative air pressure within the hollow internal chamber.

In addition to one or more of the features described herein, or as an alternative, further embodiments of the frame may include, a plurality of openings, one or more ribs, including a center rib, and a center portion. In addition to one or more of the features described herein, or as an alternative, further embodiments of the frame may include, the plurality of openings in the frame include two openings, three openings, or four openings. In addition to one or more of the features described herein, or as an alternative, further embodiments of the frame may include, a first and second opening being equal in size, a third opening being smaller in size than the first and second openings, and a fourth opening being smaller in size than the third opening. In addition to one or more of the features described herein, or as an alternative, further embodiments of the frame may include the first, second, third, and fourth openings having circular geometric shapes, or the first, second, third, and fourth openings having non-circular geometric shapes, or the first, second, third, and fourth openings having any combination of circular geometric shapes and non-circular geometric shapes.

In addition to one or more of the features described herein, or as an alternative, further embodiments of the deformable container may include, a plurality of ridges formed on an outer surface of the deformable container. The plurality of ridges correspond to the plurality of openings in the frame. In addition to one or more of the features described herein, or as an alternative, further embodiments of the deformable container may include, a plurality of surfaces, defined by the plurality of ridges. In addition to one or more of the features described herein, or as an alternative, further embodiments of the deformable container may include, the plurality of surfaces being raised and having a circular geometric shape or a non-circular geometric shape. In addition to one or more of the features described herein, or as an alternative, further embodiments of the deformable container may include, one or more of the plurality of raised surfaces are configured to be pressed inward to generate a negative air pressure within the hollow internal chamber. In addition to one or more of the features described herein, or as an alternative, further embodiments of the deformable container may include, the deformable container reversibly attaching to the frame.

According to one aspect of the disclosure of a wearable breast pump is provided. The breast pump includes a deformable container with a hollow internal chamber, the deformable container being squeezable to generate negative air pressure within the hollow internal chamber, and a frame. The frame includes a plurality of openings, a center rib, and a center portion.

In addition to one or more of the features described herein, or as an alternative, further embodiments of the frame may include, the center portion of the frame preventing a front portion of the deformable container, located behind the center portion, from collapsing inward. In addition to one or more of the features described herein, or as an alternative, further embodiments of the frame may include, a stopper on a top end of the deformable container, a flange adjacent to the stopper, a breast receiving surface on the rear portion that comes into contact with the user's breast, an aperture located on the breast receiving surface, and a hollow internal chamber. A user's mammary papilla and/or areola area is inserted at least partially into the aperture and expressed milk is collected in the hollow internal chamber.

According to one aspect of the disclosure of a wearable breast pump is provided. The breast pump includes a deformable container having a front portion, a rear portion, a top portion, and a hollow internal chamber, the deformable container being squeezable to generate negative air pressure within the hollow internal chamber, and a frame. The front portion of the deformable container has a convex outer surface and the rear portion of the deformable container is shaped to contour to a user's breast and includes an aperture passing from an outer surface of the rear portion to the hollow internal chamber. The top portion of the deformable container includes a stopper that is reversibly received in a hole formed in the top portion, with the hole passing from an outer surface of the top portion into the hollow internal chamber. The frame is configured to engage with the front portion of the deformable container and includes an outer frame portion, a center frame portion, and a plurality of openings formed between ribs that connect the center frame portion and outer frame portion. The openings are configured as receiving areas for the front portion of the deformable container.

In addition to one or more of the features described herein, or as an alternative, further embodiments of the frame may include, the center portion of the frame preventing a front portion of the deformable container, located behind the center portion, from collapsing inward. In addition to one or more of the features described herein, or as an alternative, further embodiments of the deformable container may include, a stopper on a top end of the deformable container, a flange adjacent to the stopper, a breast receiving surface on the rear portion that comes into contact with the user's breast, an aperture located on the breast receiving surface, and an hollow internal chamber. A user's mammary papilla and/or areola area is inserted at least partially into the aperture and expressed milk is collected in the hollow internal chamber.

In addition to one or more of the features described herein, or as an alternative, further embodiments of the device may include, the center frame portion of the frame prevents at least a portion of the front portion of the deformable container, located behind the center portion, from collapsing inward. In addition to one or more of the features described herein, or as an alternative, further embodiments of the device may include, the outer frame portion encircles the deformable container at a boundary between the front portion and the rear portion of the deformable container. In addition to one or more of the features described herein, or as an alternative, further embodiments of the device may include, the plurality of ribs and center frame portion defining a Y-shape. The plurality of openings expose the top portion of the deformable container and the left and right lateral sides of the front portion of the deformable container.

These and other advantages and features will become more apparent from the following description taken in conjunction with the drawings.

### BRIEF DESCRIPTION OF THE DRAWINGS

The following drawings are designed to illustrate rather than define the limits of the invention.

FIG. 1 is a front-top perspective view of an example assembled breast pump system in accordance with some embodiments taught herein.

FIG. 2 is a rear-bottom perspective view of an example assembled breast pump system in accordance with some embodiments taught herein.

FIG. 3 is a top view of an example assembled breast pump system in accordance with some embodiments taught herein.

FIG. 4 is a bottom view of an example assembled breast pump system in accordance with some embodiments taught herein.

FIG. 5 is a rear view of an example assembled breast pump system in accordance with some embodiments taught herein.

FIG. 6 is a front view of an example assembled breast pump system in accordance with some embodiments taught herein.

FIG. 7 is a side view of an example assembled breast pump system in accordance with some embodiments taught herein.

FIG. 8 is a side view, opposite of that of FIG. 7, of an example assembled breast pump system in accordance with some embodiments taught herein.

FIG. 9 is a front-top perspective view of an example dis-assembled breast pump system in accordance with some embodiments taught herein.

FIG. 10 is a cross-sectional side view of an example assembled breast pump system in accordance with some embodiments taught herein.

### DETAILED DESCRIPTION

In the present invention generally relates to a breast pump system. Specifically, embodiments of the present invention relate to a manually operated breast pump system that conforms to the user's breast.

According to an embodiment of the present invention, the breast pump system described herein comprises a pump/collector 102 for engagement with the user's breast and collection of milk expressed from the user and a frame 116 for receiving at least a portion of the pump/collector 102. The pump/collector 102 is removable from the frame 116. A hollow chamber is provided with an aperture 132 to engage at least the mammary papilla and areola area of the breast in an essentially airtight manner. The pump/collector 102 is manually operated, pressurizing and depressurizing the chamber during operation and the aperture 132 of the breast receiving surface 134 is shaped large enough for essentially only the mammary papilla and areola area to extend through. The pump/collector 102 is made of an flexible or malleable material capable of being squeezed to create pressure in the chamber. These changes in chamber pressure which stimulate lactation are transmitted to the breast via oscillations in the pump/collector 102 wall. The wall and the size of the aperture 132 prevents the breast from being pulled into the chamber, thereby mitigating painful distention during lactation. The assembled breast pump system 100 is able to be worn inside the user's bra or other supportive garment, at least in part, to enable the user to use the breast pump system 100 and collect breast milk while performing other tasks. Wearing the breast pump system 100 inside of a bra or other supportive garment also enables generally hands-free use of the breast pump system 100, apart from the deforming of the pump/collector 102 to impart the vacuum seal. The user's bra or other supportive garment can provide additional support and protection to secure the pump in place to prevent the vacuum seal being disrupted. Prior to inserting the assembled breast pump system 100 into their bra other supportive garment, the user may deform the pump/collector 102, place their mammary papilla and areola area into the aperture 132, and release the pump/collector 102 to create the vacuum seal. Alternatively, the user can insert the assembled breast pump system 100 into the bra or other supportive garment place their mammary papilla and areola area into the aperture 132, and squeeze the bra or other supportive garment to deform the pump/collector 102 to create the vacuum seal. Thus, the vacuum seal can be created prior to the user inserting the assembled breast pump system 100 into their bra or other supportive garment or after.

Additionally, the assembled breast pump system 100 can be used as a milk corrector or light-pressure pump, as opposed to a strong pressure pump for users to collect excess milk when not feeding. The wear-ability of the assembled breast pump system 100 allows the device to be worn at night while the user is sleeping to collect the excess milk. Several sizes of the wearable breast pump system 100 are available for the user to select from based on their breast shape, size, as well as mammary papilla/areola size and position.

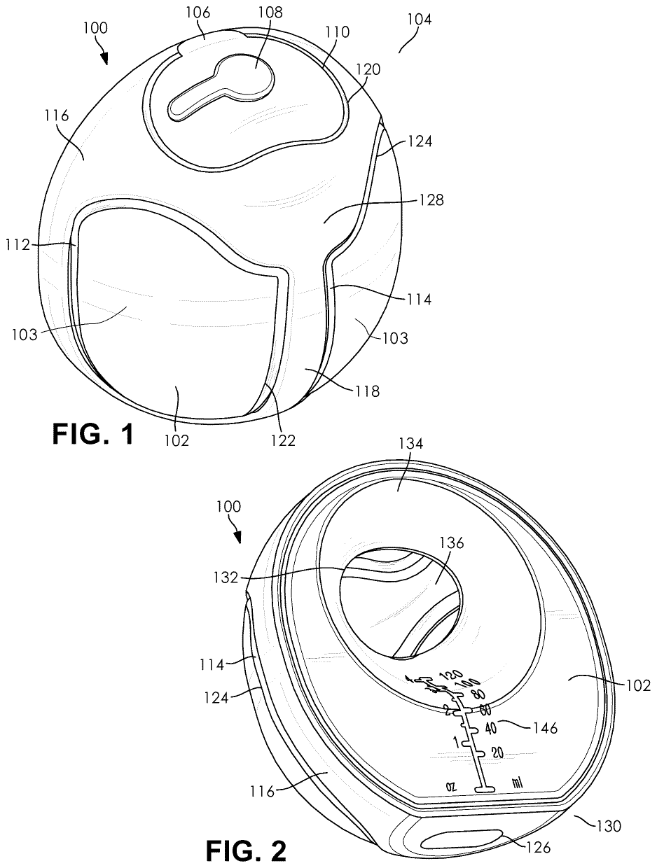

Referring to FIG. 1, the assembled breast pump system 100 includes a pump/collector 102 shaped like a breast to contour against a user's breast, and a frame 116. This shape of the pump/collector 102 provides larger milk storage capacity than traditional breast pumps, as well as surface that enables a stronger and more comfortable grip. In a preferred embodiment, the pump/collector 102 can be made of food-grade silicone. However, the pump/collector 102 could be made of any suitably flexible material that would be obvious to one of ordinary skill in the art, including, but not limited to, various rubber and plastic materials. The thickness of the material selected for the pump/collector 102 or the hardness of the material selected for the pump/collector 102, or a combination thereof, can be increased to improve the pressure formed in the chamber 136. The frame 116 receives a portion of the pump/collector 102. The pump/collector 102 is directly attachable or removable from the frame 116 to be used by a user or to be disassembled (see FIG. 9). The pump/collector 102 and frame 116 may be removed or attached together via a friction-fit, a snap-fit, or one press action, or any other release mechanism. The pump/collector 102 includes a chamber 136 (see FIG. 2) for collecting the expressed milk and tab 108 located on a top end 104 of the assembled breast pump system 100. In an embodiment, the chamber 136 can hold 3-4 ounces inclusive, 4-5 ounces inclusive, 5-6 ounces inclusive, or the like of expressed milk. However, the pump/collector 102 can possess a larger or smaller volume for holding the user's milk-producing quantity and/or their breast size.

Additionally, the frame 116 is formed from a material having the desired strength and rigidity to receive and support the pump/collector 102, including, but not limited to, a plastic material or polypropylene (PP). While the frame 116 is sufficiently rigid to receive and support the pump/collector 102, it does possess a degree of elasticity such that it can bend or twist without permanently deforming. This provides increased durability to prevent the frame 116 from breaking, as well as providing pliability for when the pump/collector 102 is filling with expressed milk. The frame 116 further includes a plurality of openings to permit portions of the pump/collector to be exposed and accessible outside of the frame 116. In an embodiment, the frame 116 can include one opening. In another embodiment, the frame 116 can include two openings. In another embodiment, the frame 116 can include three openings. In yet another embodiment, the frame 116 can include four openings. In a preferred embodiment, the frame 116 can include a first opening 120 located on the top end 104, encompassing the tab 108. In some embodiments, the frame includes a center rib 118. In some embodiments, the center rib 118 serves to divide the frame 116 into a second opening 122 and a third opening 124 located on either side of the center rib 118 of the frame 116. In some embodiments, the second and third openings 122, 124 can constitute 20%, 30%, 40%, 50%, or the like of the surface area of the frame 116. In some embodiments, the frame 116 may also include a fourth opening 126 (see FIG. 2) formed in a bottom end 130 of the frame 116. A center portion 128 of the frame 116 is formed between the first, the second, and the third openings 120, 122, 124. The first, the second, and the third openings 120, 122, 124 are positioned such that the front portion of the frame 116 is substantially T-shaped or Y-shaped, with the frame 116 extending around the circumference of the pump/collector 102. In an embodiment, the center portion 128 is positioned roughly in line, in other words in front of, with the aperture 132 of the pump/collector 102. The center portion 128 of the frame 116 may be thicker than the rest of the frame 116 to provide additional protection and shielding of the user's mammary papilla and areola area. As an illustrative example, the thicker center portion 128 of the frame 116 can resist and/or prevent the pump/collector 102 from being collapsed into or pressed against the user's mammary papilla and areola area when the user is pressurizing and depressurizing the assembled breast pump system 100.

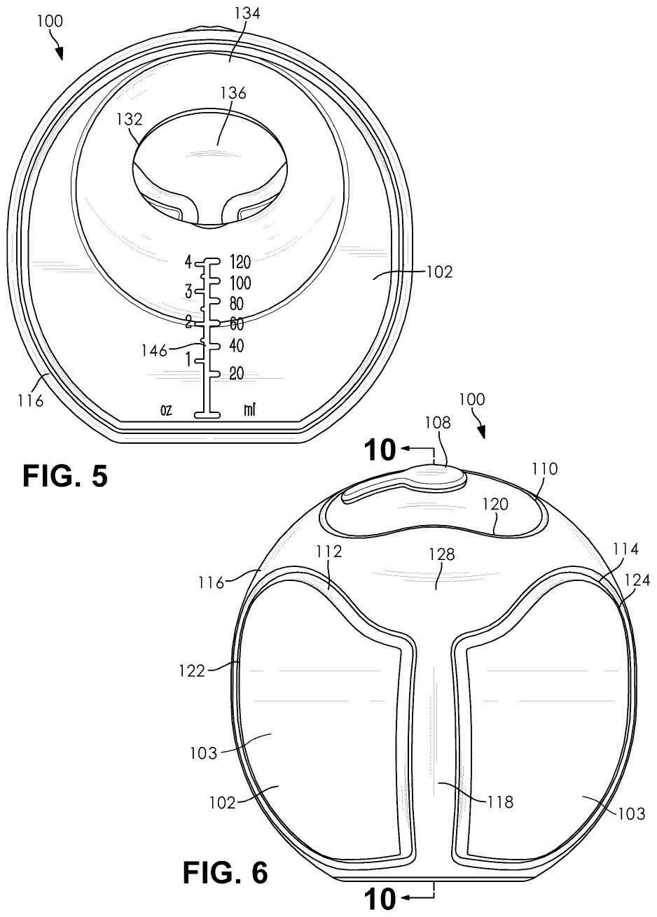

Referring to FIGS. 2 and 5, the backside of the pump/collector 102 is placed against the user's breast/chest. Unlike other wearable breast pumps, the only component of the breast pump system 100 that comes into contact with the user's expressed milk during normal use is the chamber 136 and the aperture 132. As an illustrative example, during normal use, milk is intended to flow toward the aperture 132 and then fill the chamber 136. During normal use, milk does not flow through any parts of the housing, for maximum hygiene and ease of cleaning. The backside of the pump/collector 102 can be rounded, substantially flat with flexibility, or flat. The breast receiving surface 134 can transverse the entire backside of the pump/collector 102 or can transverse only a portion of the backside of the pump/collector 102. The breast receiving surface 134 shape may be circular or non-circular. For example, the breast receiving surface 134 shape can be circular, egg, oval, ovoid, or stadium. In the embodiment where the breast receiving surface 134 transverses a portion of the backside of the pump/collector 102, the breast receiving surface 134 can be positioned substantially center of the backside, off-center towards the bottom end 130, or off-center towards the top end 104. In an embodiment, the breast receiving surface 134 can be part of a continuous surface of the pump/collector 102. In another embodiment, the breast receiving surface 134 can be concave, with the apex located within the chamber 136, such that the breast receiving surface 134 cups the user's breast. In an embodiment, the backside of the pump/collector can include volume indicators 146 for the user to track how much milk has been expressed.

The aperture 132 is positioned within the breast receiving surface 134. In a preferred embodiment, the aperture 132 has a concentric shape and is large enough to permit at least a portion of the mammary papilla and areola area to extend through the aperture 132 and small enough that a vacuum seal with the breast is formed during use. The aperture 132 shape may be circular or non-circular. For example, the breast receiving surface 134 shape can be circular, reuleaux triangle, egg, oval, ovoid, or stadium. Additionally, the frame 116 at the bottom end 130 of the assembled breast pump system 100 can be flat. In this embodiment, the flat end allows for the assembled breast pump system 100 to be placed on a surface after use without it tipping over and spilling the expressed milk.

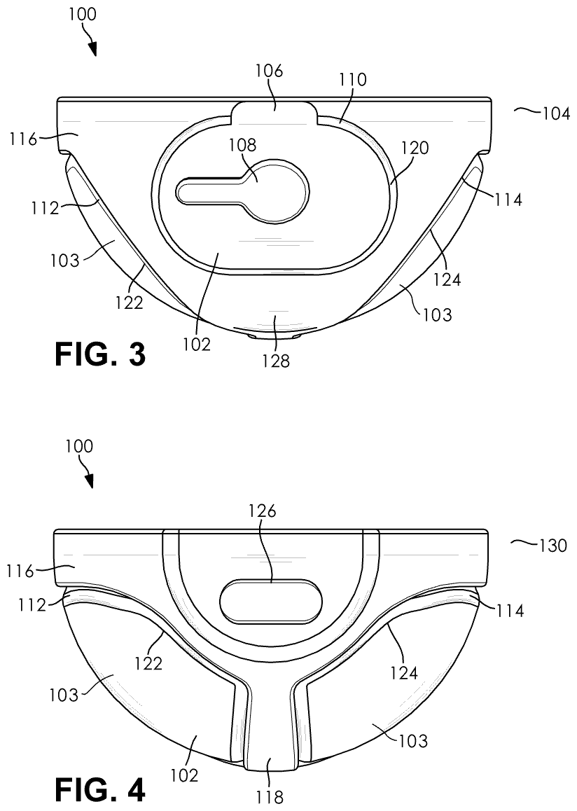

Referring to FIGS. 3 and 4, the pump/collector 102 includes a plurality of ridges. The plurality of ridges and raised surfaces 140 coincide with the plurality of openings in the frame 116. In an embodiment, the plurality of ridges define one or more raised surfaces 140 on the pump/collector 102 that reversibly couple with the plurality of openings. The plurality of ridges and raised surfaces provide an improved connection between the pump/collector 102 and the frame 116. As shown in FIGS. 3 and 4, a first ridge 110 couples with the first opening 120, a second ridge 112 couples with the second opening 122, and a third ridge 114 couples with the third opening 124. In another embodiment, a forth ridge (not pictured) couples with the fourth opening 126. The shape of the plurality of ridges and raised surfaces substantially correspond to the shape of the opening with which the ridge and raised surface couples. Alternatively, the shape of the plurality of ridges and raised surfaces can be considered to define one or more channels, grooves, or indentations in the surface of the pump/collector 102 that are collectively configured to receive the frame.

The unique geometries of the openings and their placement in the frame 116 of the present disclosure can enable improved pressurization and depressurization in the chamber 136, as well as improve the user experience by reducing unnecessary pressure on the user's breast, mammary papilla, and areola area and mitigating painful distention. Referring to FIG. 6, in an embodiment, the first, the second, the third, and the fourth openings 120, 122, 124, 126 can be equal in size. In another embodiment, the first, the second, and the third openings 120, 122, 124 can be equal in size and the fourth opening 126 is smaller in size than the first, the second, and the third openings 120, 122, 124. In another embodiment, the first, the second, the third, and the fourth openings 120, 122, 124, 126 can all be unequal in size. In another embodiment, the second and the third openings 122, 124 can be equal in size, the first opening 120 can be smaller than the second and the third openings 122, 124, and the fourth opening 126 can be smaller than the first opening 120. In this embodiment, shape of the openings are not limited to a single circular shape or non-circular shape. For example, the first opening 120 shape can be circular, egg, oval, ovoid, pillow, peanut, or two-lobed, while the second and the third openings 122, 124 can be circular, egg, oval, ovoid, or indented oval.

When using the assembled breast pump system 100, the user's mammary papilla and areola area is positioned within the aperture 132 and their breast is placed against the breast receiving surface 134. Referring to FIGS. 5 and 6, to express the milk and begin lactation, the areas of the pump/collector 102 exposed in the second and the third openings 122, 124 are squeezed/pumped/pushed and released repeatedly. The pump/collector 102 not only helps to support the breast during use, but oscillations of its walls also press and suck upon the area surrounding the mammary papilla and areola area as done by a nursing baby. Rather than a multi-chamber device with a complicated pumping system, all that is necessary is the single chamber 136 which may be pressurized and depressurized by means of applying pressure to the pump/collector 102. When the air is squeezed out of the chamber 136 of the pump/collector 102, a vacuum seal is created with the breast. The aperture 132 and breast receiving surface 134 are designed to create a seal between the assembled breast pump system 100 and the user's breast under negative pressure to reduce the risk of spilling the expressed milk. The pump/collector 102 will try to return to its original state (i.e. un-squeezed and expanded), and hence, causes a “pull” or pressure inside the pump/collector 102 that in turn causes the milk to flow out from the mammary papilla.

By applying pressure on the sides 103 of the pump/collector 102, as opposed to applying pressing on the front, the pump/collector 102 does not rub the mammary papilla, thereby reducing discomfort and pain during use. Additionally, applying pressure on two points of contact dispels more air from the chamber 136 and improves the milk collection; thus requiring fewer pumps. The center rib 118 of the frame 116 provides support to the pump/collector 102 during use and helps prevent the pump/collector 102 denting or collapsing inward (i.e. folding toward the mammary papilla) during use.

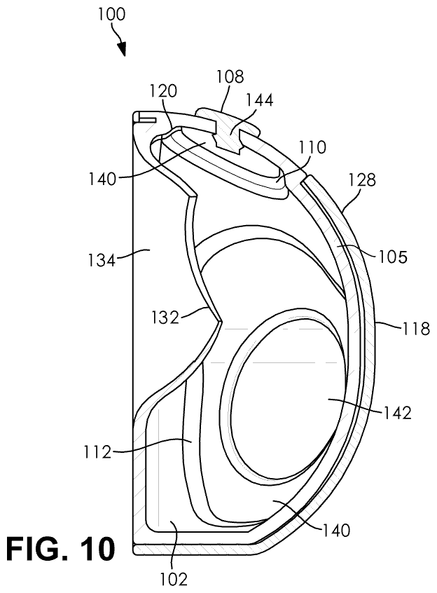

When the user has filled the pump/collector 102 or is simply done pumping, the tab 108 is pulled up, revealing a hole passing from an outer surface of the pump/collector 102 into the hollow internal chamber of the pump/collector 102. Removing the tab 108 from the hole allows the chamber 136 to depressurize and release the vacuum seal. Once this is done, the user may remove the assembled breast pump system 100 from their person. The tab 108 can include a stopper/plug 144 (see FIG. 10) adapted to securely fix into the hole for an air tight fit. The tab 108 can further include an extension or tail that is secured to the pump/collector 102 such that when the tab 108 is released it remains attached the pump/collector 102. In addition to releasing the vacuum seal, removing the tab 108 from the hole provides a pathway for milk to be emptied from the pump/collector 102.

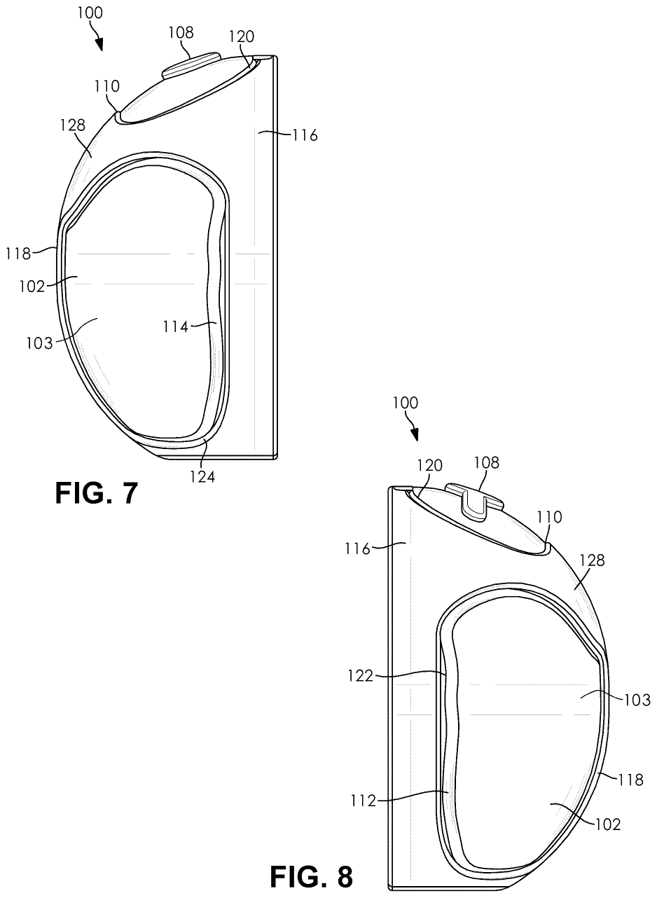

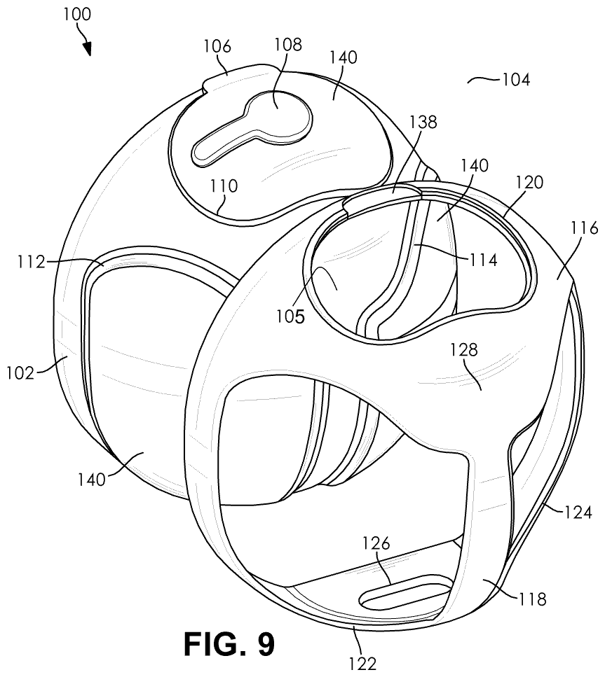

FIGS. 7 and 8 show side views of the breast pump system described above. The frame 116 encompasses the circumference of the pump/collector 102 to support the pump/collector 102 and provide a stronger and more comfortable grip.

Referring to FIG. 9, the modularity of the breast pump system 100 allows for easy assembly, disassembly, as well as replacement of different parts if needed. The modularity further allows for the components to be easily washed and/or sterilized. The pump/collector 102, of which is in contact with milk during pumping, may be efficiently and easily cleaned. As shown in FIG. 9, and described above, the pump/collector 102 includes a plurality of raised ridges for coupling with the openings of the frame 116. The surface of the pump/collector encompassed by the raised ridge, that is exposed through the opening, can be a raised surface 140. The frame 116 further includes an indent 138 for receiving the flange 106 of the pump/collector 102. The plurality of raised ridges and the flange 106 helps secure the pump/collector 102 and the frame 116.

To assemble the pump/collector 102 and frame 116, the tab 108 is inserted into a hole in the first opening 120. Light pressure is applied to the middle and lower half of the pump/collector 102 to slowly insert the lower half of the pump/collector 102 into the frame 116.

Referring to FIG. 10, another embodiment of the assemble breast pump system 100 is depicted. In this embodiment, the assembled breast pump system 100, as described above, includes a second raised surface 142 to aid in pressurizing the chamber 136. The second raised surfaces 142 is located within the first raised surfaces 140, forming a concentric shape, and is also exposed through the opening of the frame 116. In an embodiment, the second raised surfaces 142 are located on the first raised surfaces 140 that extend through the second and third openings 122, 124, of which pressure is applied during use. The shape of the second raised surfaces 142 are not limited to a single circular shape or non-circular shape. For example, the second raised surfaces 142 shape can be circular, egg, oval, ovoid, indented oval, pillow, peanut, or two-lobed. The shape of the second raised surfaces 142 allow for more air to be expelled from the chamber 136, thereby increasing the amount of milk expressed with each pump.

According to an alternative embodiment of the present invention, the breast pump system comprises a pump/collector 102 for engagement with the user's breast and collection of milk expressed from the user without a frame 116 to provide a one-piece design. The frameless embodiment may be made from a single material or multiple materials, as described below. Similar to the pump/collector 102 described above, the pump/collector 102 of the frameless embodiment, includes a hollow chamber provided with an aperture 132 to engage at least the mammary papilla and areola area of the breast in an essentially airtight manner. The pump/collector 102 is manually operated, pressurizing and depressurizing the chamber during operation and the aperture 132 of the breast receiving surface 134 is shaped large enough for essentially only the mammary papilla and areola area to extend through. The pump/collector 102 is made of an flexible or malleable material capable of squeezed and released to create pressure in the chamber. These changes in chamber pressure which stimulate lactation are transmitted to the breast via oscillations in the pump/collector 102 wall. The wall and the size of the aperture 132 prevents the breast from being pulled into the chamber, thereby mitigating painful distention during lactation.

In a frameless embodiment of the invention, the middle and center front portion 105 of the pump/collector 102 material may be more rigid or supporting than the rest of the pump/collector 102 and in particular more rigid or supporting than the side portions 103 of the pump/collector 102. The middle and center front portion 105 of the pump/collector 102 material may be made more rigid or supportive by (i) being made of a harder or less flexible material than other portions of the pump/collector 102, (ii) being made thicker than other portions of the pump/collector 102, or (iii) a combination thereof. The middle and center front portion 105 of the pump/collector 102 may be substantially T-shaped or Y-shaped bordering the side portions 103. The middle and center front portion 105 is positioned roughly in line with, in other words in front of, the aperture 132 of the pump/collector 102. The increased thickness and/or hardness of the material of middle and center front portion 105 of the pump/collector 102 provides additional protection and shielding of the user's mammary papilla and areola area. The middle and center front portion 105 divides the pump/collector 120 into a first and second raised surfaces 140, to be pressed during use. As an illustrative example, the thicker middle and center front portion 105 of the pump/collector 102 can resist and/or prevent the pump/collector 102 from being collapsed into or pressed against the user's mammary papilla and areola area when the user is pressurizing and depressurizing the breast pump system 100.

The assembled breast pump system 100 is able to be worn inside the user's bra or other supportive garment, at least in part, to enable the user to use the breast pump system 100 and collect breast milk while performing other tasks. Wearing the breast pump system 100 inside of a bra or other supportive garment also enables generally hands-free use of the breast pump system 100, apart from the deforming of the pump/collector 102 to impart the vacuum seal. The user's bra or other supportive garment can provide additional support and protection to secure the pump in place to prevent the vacuum seal being disrupted. Prior to inserting the breast pump system 100 into their bra or other supportive garment, the user may deform the pump/collector 102, place their mammary papilla and areola area into the aperture 132, and release the pump/collector 102 to create the vacuum seal. Alternatively, the user can insert the breast pump system 100 into the bra place their mammary papilla and areola area into the aperture 132, and squeeze the bra to deform the pump/collector 102 to create the vacuum seal. Thus, the vacuum seal can be created prior to the user inserting the breast pump system 100 into their bra or after. Additionally, the breast pump system 100 can be used as a milk corrector or light-pressure pump, as opposed to a strong pressure pump for users to collect excess milk when not feeding. The wear-ability of the breast pump system 100 allows the device to be worn at night while the user is sleeping to collect the excess milk. Several sizes of the wearable breast pump system 100 are available for the user to select from based on their breast shape, size, as well as mammary papilla/areola size and position.

The breast pump system 100 includes a pump/collector 102 shaped like a breast to contour against a user's breast. This shape of the pump/collector 102 provides larger milk storage capacity than traditional breast pumps, as well as surface that enables a stronger and more comfortable grip. The pump/collector 102 includes a chamber 136 (see FIG. 2) for collecting the expressed milk and tab 108 located on a top end 104 of the breast pump system 100. In an embodiment, the chamber 136 can hold 3-4 ounces inclusive, 4-5 ounces inclusive, 5-6 ounces inclusive, or the like of expressed milk. However, the pump/collector 102 can possess a larger or smaller volume for holding the user's milk-producing quantity and/or their breast size.

The backside of the pump/collector 102 is placed against the user's breast/chest. Unlike other wearable breast pumps, the only component of the breast pump system 100 that comes into contact with the user's expressed milk during normal use is the chamber 136 and the aperture 32. As an illustrative example, during normal use, milk is intended to flow toward the aperture 132 and then fill the chamber 136. During normal use, milk does not flow through any parts of the housing, for maximum hygiene and ease of cleaning. The backside of the pump/collector 102 can be rounded, substantially flat with flexibility, or flat. The breast receiving surface 134 can transverse the entire backside of the pump/collector 102 or can transverse only a portion of the backside of the pump/collector 102. The breast receiving surface 134 shape may be circular or non-circular. For example, the breast receiving surface 134 shape can be circular, egg, oval, ovoid, or stadium. In the embodiment where the breast receiving surface 134 transverses a portion of the backside of the pump/collector 102, the breast receiving surface 134 can be positioned substantially center of the backside, off-center towards the bottom end 130, or off-center towards the top end 104. In an embodiment, the breast receiving surface 134 can be part of a continuous surface of the pump/collector 102. In another embodiment, the breast receiving surface 134 can be concave, with the apex located within the chamber 136, such that the breast receiving surface 134 cups the user's breast. In an embodiment, the backside of the pump/collector can include volume indicators 146 for the user to track how much milk has been expressed.

The aperture 132 is positioned within the breast receiving surface 134. In a preferred embodiment, the aperture 132 has a concentric shape and is large enough to permit at least a portion of the mammary papilla and areola area to extend through the aperture 132 and small enough that a vacuum seal with the breast is formed during use. The aperture 132 shape may be circular or non-circular. For example, the breast receiving surface 134 shape can be circular, reuleaux triangle, egg, oval, ovoid, or stadium. Additionally, the pump/collector 102 at the bottom end 130 of the breast pump system 100 can be flat. The pump/collector 102 material may also be thicker or harder at the bottom end 130. In this embodiment, the flat end allows for the breast pump system 100 to be placed on a surface after use without it tipping over and spilling the expressed milk.

In a preferred embodiment, the pump/collector 102 can be made of food-grade silicone. However, the pump/collector 102 could be made of any suitably flexible material that would be obvious to one of ordinary skill in the art, including, but not limited to, various rubber and plastic materials. The thickness of the material selected for the pump/collector 102 or the hardness of the material selected for the pump/collector 102, or a combination thereof, can be adapted to regulate the pressure formed in the chamber 136.

According to an embodiment of the invention, the shape of the first and second raised surfaces 140 are not limited to a single circular shape or non-circular shape. For example, the first and second surfaces 140 can be circular, egg, oval, ovoid, or indented oval. The unique geometries of the raised surfaces 140 and their placement on the surface area of the pump/collector 102 of the present disclosure can enable improved pressurization and depressurization in the chamber 136, as well as improve the user experience by reducing unnecessary pressure on the user's breast, mammary papilla, and areola area and mitigating painful distention.

When using the breast pump system 100, the user's mammary papilla and areola area is positioned within the aperture 132 and their breast is placed against the breast receiving surface 134. To express the milk and begin lactation, the first and second raised surfaces 140 of the pump/collector 102 are squeezed/pumped/pushed and released repeatedly. The pump/collector 102 not only helps to support the breast during use, but oscillations of its walls also press and suck upon the area surrounding the mammary papilla and areola area as done by a nursing baby. Rather than a multi-chamber device with a complicated pumping system, all that is necessary is the single chamber 136 which may be pressurized and depressurized by means of applying pressure to the pump/collector 102. When the air is squeezed out of the chamber 136 of the pump/collector 102, a vacuum seal is created with the breast. The aperture 132 and breast receiving surface 134 are designed to create a seal between the breast pump system 100 and the user's breast under negative pressure to reduce the risk of spilling the expressed milk. The pump/collector 102 will try to return to its original state (i.e. un-squeezed and expanded), and hence, causes a “pull” or pressure inside the pump/collector 102 that in turn causes the milk to flow out from the mammary papilla.

By applying pressure on the sides 103 of the pump/collector 102, as opposed to applying pressing on the front, the pump/collector 102 does not rub the mammary papilla, thereby reducing discomfort and pain during use. Additionally, applying pressure on two points of contact dispels more air from the chamber 136 and improves the milk collection, which in turn requires fewer pumps. The middle portion of the pump/collector 102 provides support to the pump/collector 102 during use and helps prevent the pump/collector 102 denting or collapsing inward (i.e. folding toward the mammary papilla) during use.

When the user has filled the pump/collector 102 or is simply done pumping, the tab 108 is pulled up, revealing a hole passing from an outer surface of the pump/collector 102 into the hollow internal chamber of the pump/collector 102. Removing the tab 108 from the hole allows the chamber 136 to depressurize and release the vacuum seal. Once this is done, the user may remove the breast pump system 100 from their person. The tab 108 can include a stopper/plug 144 (see FIG. 10) adapted to securely fix into the hole for an air tight fit. The tab 108 can further include an extension or tail that is secured to the pump/collector 102 such that when the tab 108 is released it remains attached the pump/collector 102. In addition to releasing the vacuum seal, removing the tab 108 from the hole provides a pathway for milk to be emptied from the pump/collector 102.

In this embodiment, a secondary raised surface located within the first and second raised surfaces 140 can be included, to aid in pressurizing the chamber 136. The secondary raised surfaces 142 are located within the first and second raised surfaces 140, forming a concentric shape. In an embodiment, the secondary raised surfaces 142 are located on the first and second raised surfaces 140, of which pressure is applied during use. The shape of the secondary raised surfaces 142 are not limited to a single circular shape or non-circular shape. For example, the secondary raised surfaces 142 shape can be circular, egg, oval, ovoid, indented oval, pillow, peanut, or two-lobed. The shape of the secondary raised surfaces 142 allow for more air to be expelled from the chamber 136, thereby increasing the amount of milk expressed with each pump.

Unless defined otherwise, all technical and scientific terms used herein have the same meaning as commonly understood by one of ordinary skill in the art to which this disclosure belongs. It is also to be understood that the terminology used herein is for the purpose of describing particular embodiments only, and is not intended to be limiting, since the scope of the present disclosure will be limited only by the appended claims. Although any methods and materials similar or equivalent to those taught herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

1. A breast pump system comprising:
a deformable container having a front portion and a rear portion, wherein the rear portion of the deformable container is shaped to contour a user's breast; and
a frame that is configured to engage with the front portion of the deformable container.

2. The breast pump system of claim 1, the deformable container further comprising:
a stopper located at a top end of the deformable container;
a flange located adjacent to the stopper at the top end of the deformable container;
a hollow internal chamber for receiving expressed milk;
a breast receiving surface located on the rear portion of the deformable container; and
an aperture located on the breast receiving surface, the aperture configured to receive a mammary papilla and/or areola area of a user.

3. The breast pump system of claim 2, wherein the deformable container is squeezable to generate negative air pressure within the hollow internal chamber.

4. The breast pump system of claim 1, the frame further comprises:
a plurality of openings;
a center rib;
and a center portion.

5. The breast pump system of claim 4, wherein the plurality of openings include two openings, three openings, or four openings.

6. The breast pump system of claim 5, wherein a first and a second openings are equal in size, a third opening is smaller in size than the first and the second openings, and a fourth opening is smaller in size than the third opening.

7. The breast pump system of claim 6, wherein the first, the second, the third, and the fourth opening have a circular or non-circular geometric shape.

8. The breast pump system of claim 4, further comprising a plurality of ridges formed on an outer surface of the deformable container, wherein the plurality of ridges correspond to the plurality of openings.

9. The breast pump system of claim 8, wherein the plurality of ridges define a plurality of surfaces.

10. The breast pump system of claim 9, wherein the plurality of surfaces are raised, having a circular geometric shape or a non-circular geometric shape.

11. The breast pump system of claim 10, wherein one or more of the plurality of surfaces are configured to be pressed inward to generate a negative air pressure within a hollow internal chamber of the deformable container.

12. The breast pump system of claim 4, wherein at least the center portion of the frame prevents the front portion of the deformable container that is behind the center portion of the frame from collapsing inward.

13. The breast pump system of claim 1, wherein the deformable container reversibly attaches to the frame.

14. A breast pump system comprising:
a deformable container having a hollow internal chamber, wherein the deformable container is squeezable to generate negative air pressure within the hollow chamber; and
a frame comprising:
a plurality of openings;
a center rib;
and a center portion.

15. The breast pump system of claim 14, wherein at least the center portion of the frame prevents a front portion of the deformable container that is behind the center portion of the frame from collapsing inward.

16. The breast pump system of claim 14, further comprising:
a stopper located at a top end of the deformable container;
a flange located adjacent to the stopper at the top end of the deformable container;
a breast receiving surface located on a backside of the deformable container; and
an aperture located on the breast receiving surface, the aperture configured to receive a mammary papilla and/or areola area of a user.

17. A breast pump system comprising:
a deformable container having a hollow internal chamber that is squeezable to generate negative air pressure within the hollow chamber, the deformable container comprising:
a front portion having a convex outer surface,
a rear portion shaped to contour to a breast and formed with an aperture, wherein the aperture passes from an outer surface of the rear portion to the hollow internal chamber, and
a top portion having a stopper reversibly received in a hole formed in the top portion, wherein the hole passes from an outer surface of the top portion to the hollow internal chamber; and
a frame that is configured to engage with the front portion of the deformable container, the frame comprising:
an outer frame portion,
a center frame portion, and
a plurality of openings between ribs connecting between the center frame portion and outer frame portion, wherein the outer frame portion, center frame portion, and plurality of ribs define a plurality of openings through the frame, the plurality of openings being configured to receive areas of the front portion of the deformable container.

18. The breast pump system of claim 17, wherein the center frame portion is configured to prevent at least a portion of the front portion of the deformable container from collapsing inward.

19. The breast pump system of claim 17, wherein the outer frame portion encircles the deformable container at a boundary between the front portion and the rear portion of the deformable container.

20. The breast pump system of claim 19, wherein the plurality of ribs and center frame portion define a Y-shape and the plurality of openings expose the top portion of the deformable container and each lateral side of the front portion of the deformable container.
